(19) United States
(12) Patent Application Publication
(10) Pub. No.: US 2019/0357950 A1
(43) Pub. Date: Nov. 28, 2019

(54) SURGICAL IMPLANT SYSTEM FOR TREATING FIFTH METATARSAL JONES FRACTURES

(71) Applicant: The Methodist Hospital, Houston, TX (US)

(72) Inventors: Derek T. Bernstein, Philadelphia, PA (US); Bradley K. Weiner, Houston, TX (US)

(21) Appl. No.: 16/485,037

(22) PCT Filed: Feb. 9, 2018
(86) PCT No.: PCT/US18/17593

§ 371 Date: Aug. 9, 2019

Related U.S. Application Data
(60) Provisional application No. 62/457,607, filed on Feb. 10, 2017.

(51) Int. Cl. A61B 17/72; A61B 17/80; A61B 17/86

(52) U.S. Cl. CPC A61B 17/7291; A61B 17/863; A61B 17/809; A61B 17/8061

(57) ABSTRACT

A surgical implant system comprising: a spiked washer for impacting against the proximal end of a bone so as to achieve fixed positioning relative to the proximal end of the bone and an intramedullary screw for advancement through the washer and down the intramedullary canal of a bone. The intramedullary screw comprises a shaft and a head, distal threads formed on the shaft for engaging the wall of the intramedullary canal at the distal end of the intramedullary screw and proximal threads formed on the shaft for engaging the wall of the intramedullary canal at the proximal end of the intramedullary screw so as to generate a compressive force therebetween. The head comprises locking threads for engaging the screw threads formed in the spiked washer, whereby to lock the intramedullary screw to the spiked washer.

### REFERENCE TO PENDING PRIOR PATENT APPLICATION

This patent application claims benefit of pending prior U.S. Provisional Patent Application Ser. No. 62/457,607, filed Feb. 10, 2017 by The Methodist Hospital and Derek T. Bernstein et al. for SURGICAL IMPLANT SYSTEM FOR TREATING FIFTH METATARSAL JONES FRACTURES (Attorney's Docket No. METHODIST-35 PROV), which patent application is hereby incorporated herein by reference.

### FIELD OF THE INVENTION

This invention relates to surgical apparatus in general, and more particularly to surgical apparatus for fracture fixation.

### BACKGROUND OF THE INVENTION

The fifth metatarsal bone (“the fifth metatarsal”) is a long bone located on the outside of the foot. As seen in FIG. 1, the fifth metatarsal bridges the mid- and forefoot regions. Fractures of the fifth metatarsal occur most commonly when the bone fails under tension, typically on the half of the bone closer to the heel (“the proximal half” of the bone). See FIG. 2 for examples of various fractures of the fifth metatarsal. Of particular concern is the fracture pattern known as a “Jones fracture”, which is often seen in high-level athletes who place a high level of stress on their feet during play.

Treatment of Jones fractures traditionally involves placing an intramedullary screw down the intramedullary canal of the fifth metatarsal, from the heel toward the toes. See FIG. 3, which shows a partially-threaded intramedullary screw being used to repair a Jones fracture in the conventional manner. The partially-threaded intramedullary screw has threads on only the front half of the intramedullary screw (“the distal half” of the intramedullary screw), which allows the intramedullary screw to compress the fracture site as the intramedullary screw is advanced down the intramedullary canal and the head of the intramedullary screw engages the proximal end of the fifth metatarsal. This intramedullary approach is intended to maximize healing potential inasmuch as the soft tissue and blood supply over the fracture site are minimally disturbed by surgical dissection.

Confirmation of fracture healing is generally accomplished using X-rays and/or CT scans. CT scans are generally most desirable since they typically provide a high resolution image which allows for more direct assessment of bridging callus across the fracture site with higher resolution than other imaging modalities. Once the Jones fracture has healed, athletes can return to play.

Unfortunately, despite confirmation of successful healing on a CT scan, some athletes may “re-fracture” the fifth metatarsal through the old fracture site. This may occur even with optimal intramedullary screw placement technique and position. This can occur several months after the athlete returns to full play.

Revision surgery is typically performed in these re-fracture cases. Such revision surgery can be effected either by (i) placing a new, larger screw down the intramedullary canal of the fifth metatarsal, or by (ii) using a plate and screws to plate across the Jones fracture. Using a new, larger screw is not possible in all cases and, even where it is, does not guarantee against further re-fracture. Using a plate and screws to plate across the Jones fracture requires a much larger incision, with the attendant trauma to the patient.

Bone grafting is also used and is frequently harvested from the calcaneus bone (“the heel bone”), but this requires a second incision and further bony trauma.

The consequences to the patient (particularly elite-level athletes) who must undergo a second, revision surgery can be quite severe. Many elite-level players, such as those competing at Division I schools or at the professional level, must produce on the playing field in order to maintain their position and/or salary. Thus, a prolonged medical absence can be career-threatening. Therefore, re-fracture after surgical treatment of a Jones fracture, even after CT confirmation of excellent healing, is both frustrating and perplexing for the patient and for the surgeon.

In considering why these re-fracture patients might be experiencing this complication, one must consider the anatomy of the fifth metatarsal itself. More particularly, and looking now at FIG. 4, at the proximal end of the fifth metatarsal (i.e., the portion closer to the heel), the bone is wider and the cortex (i.e., the hard outer bone) is thinner. Moving toward the toes, the width of the fifth metatarsal bone tapers and the cortex becomes thicker and harder. The screw diameter for the intramedullary screw is generally determined by the width of the intramedullary canal of the fifth metatarsal, but in any case the intramedullary screw cannot be wider than the narrowest portion of the fifth metatarsal with which it makes contact. Ideally, the intramedullary screw contacts the cortical bone from within the intramedullary canal in order to maximize purchase. This means that closer to the heel, where the fifth metatarsal is wider and weaker, the shaft of the intramedullary screw is not in direct contact with the cortical bone. Instead, the shaft of the intramedullary screw is encased in spongy cancellous bone, which provides relatively modest support for the shaft of the intramedullary screw.

Furthermore, bones tend to bend very small amounts when placed under stress. This means that when athletes run and jump, thereby putting tension on the fifth metatarsal, the fifth metatarsal naturally bends a very small amount and then springs back to its normal (i.e., unstressed) configuration. In other words, the fifth metatarsal is somewhat elastic. With the intramedullary screw in place within the fifth metatarsal, the intramedullary screw remains well fixed within the more distal, reduced-diameter portion of the intramedullary canal, but as the bone bends, the proximal end of the intramedullary screw can “windshield wiper” within the wider, weaker more proximal cancellous bone (i.e., the portion of the bone closer to the heel). With time, this relative motion between the proximal end of the fifth metatarsal and the intramedullary screw can result in a loss of fixation at the proximal end of the intramedullary screw and exaggerated bending at the fracture site, which may then re-fracture.

In addition, conventional intramedullary screws utilized in the setting of proximal fifth metatarsal fractures function by achieving compression across the fracture site to allow healing. The partially threaded nature of the intramedullary screw provides distal fixation as the screw advances across the fracture site. Since the proximal portion of the intramedullary screw lacks threads, it simply slides along the tract created by the distal aspect of the intramedullary screw. Fixation distally ultimately occurs by engagement of the screw threads with the bone. Proximally, a single screw has modest compression of the metaphyseal bone from the screw head; however, the thin cortical bone and the relative size and shape mismatch between the proximal portion of the 5^{th} metatarsal and the screw head limits the quality of fixation. It is possible, for example, to intrude the intramedullary screw (i.e., to advance the intramedullary screw too far along the intramedullary canal, so that the head of the screw actually intrudes the intramedullary canal), thereby losing proximal fixation. A simple washer more evenly distributes the force of the screw head and improves the quality of proximal compression; however, as the 5^{th} metatarsal bends under natural stress, a simple washer fails to control rotation and bending. The result is that the intramedullary screw does not conform to the bending moments experienced by the bone and the proximal portion of the screw demonstrates the “windshield wiper” effect.

Thus there is a need for a new and improved system for providing intramedullary screw fixation of a Jones fracture of the fifth metatarsal, wherein the system provides for adequate fixation of both the distal and proximal ends of the intramedullary screw.

### SUMMARY OF THE INVENTION

The present invention comprises the provision and use of a new and improved system for providing intramedullary screw fixation of a Jones fracture of the fifth metatarsal, wherein the system provides for adequate fixation of both the distal and proximal ends of the intramedullary screw.

More particularly, the present invention comprises a novel surgical implant system comprising (i) a spiked washer for impacting against the proximal end of the fifth metatarsal so as to achieve fixed positioning relative to the proximal end of the fifth metatarsal, wherein the spiked washer comprises a disk-like body having a central opening, a plurality of spikes extending distally from the disk-like body, and screw threads formed in the central opening of the disk-like body, and (ii) an intramedullary screw for advancement through the central opening of the spiked washer and down the intramedullary canal of the fifth metatarsal, wherein the intramedullary screw comprises a shaft and a head, distal threads formed on the shaft for engaging the narrowed wall of the intramedullary canal at the distal end of the intramedullary screw, proximal threads formed on the shaft for engaging the wall of the intramedullary canal at the proximal end of the intramedullary screw, and locking threads formed on the head of the intramedullary screw for engaging the screw threads formed in the central opening of the spiked washer.

As a result of this construction, the distal threads of the intramedullary screw can securely anchor the distal end of the intramedullary screw against the narrowed wall of the intramedullary canal, and the proximal threads of the intramedullary screw can securely anchor the proximal end of the intramedullary screw against the wall of the intramedullary canal, whereby to secure a distal bone fragment in position relative to a proximal bone fragment. Furthermore, when the intramedullary screw has been fully inserted into the intramedullary canal, the locking threads formed on the head of the intramedullary screw engage the screw threads formed in the central opening of the spiked washer, whereby to lock the intramedullary screw to the spiked washer. In essence, the spiked washer, which is impacted into position against the proximal end of the fifth metatarsal, serves as a capture plate for holding the proximal end of the intramedullary screw fixed in position relative to the proximal end of the fifth metatarsal, whereby to eliminate the aforementioned “windshield wiper” effect which could otherwise occur within the wider, weaker cancellous bone of the proximal end of the fifth metatarsal. And it should also be appreciated that the spiked washer of the present invention also provides rotational control (i.e., locking the implanted screw against rotation). As a result, the novel surgical implant system of the present invention can prevent the fracture site from experiencing a substantial bending moment under stress, thereby preventing re-fracture.

In one preferred form of the invention, the intramedullary screw comprises distal screw threads for engaging the distal bone fragment and proximal screw threads for engaging the proximal bone fragment. The distal screw threads and the proximal screw threads may be separated by an unthreaded region and may have different pitches, with the distal threads having a more aggressive pitch than the proximal threads, whereby to create compression between the distal bone fragment and the proximal bone fragment when the intramedullary screw is inserted into the bone. The intramedullary screw also preferably comprises locking threads disposed at the proximalmost end of the intramedullary screw for engaging corresponding threads formed on the inside surface of the central opening of the spiked washer, whereby to facilitate locking of the intramedullary screw to the spiked washer when the spiked washer is secured to the proximal end of the fifth metatarsal and the intramedullary screw is deployed through the central opening of the spiked washer and down the intramedullary canal of the fifth metatarsal. More particularly, in this form of the invention, the thread pitch on the proximal thread of the intramedullary screw is finer than the thread pitch on the distal thread of the intramedullary screw (i.e., the thread pitch on the proximal thread has more threads per inch than the thread pitch on the distal thread), whereby to provide a pitch differential which aids in reducing fractures and in creating compression between bone fragments. The spiked washer is secured to the proximal end of the fifth metatarsal and the intramedullary screw is deployed through the central opening of the spiked washer and down the intramedullary canal of the fifth metatarsal, with the locking threads of the intramedullary screw engaging the threaded central opening of the spiked washer when the intramedullary screw is fully deployed in the intramedullary canal, whereby to lock the intramedullary screw to the spiked washer.

And in one preferred form of the invention, the central opening of the spiked washer, and the screw threads formed in the central opening, are coordinated with the locking threads of the intramedullary screw so as to allow for variable-angle locking of the intramedullary screw to the spiked washer.

In use, the spiked washer is impacted into position against the proximal end of the fifth metatarsal, and then the intramedullary screw is advanced through the central opening of the spiked washer and down the intramedullary canal of the fifth metatarsal, such that the distal threads of the intramedullary screw engage with the wall of the intramedullary canal and the proximal threads of the intramedullary screw engage with the wall of the intramedullary canal, with the pitch differential between the distal and proximal threads reducing the fracture and creating compression between the bone fragments. When the intramedullary screw is fully deployed in the intramedullary canal, the locking threads at the proximalmost end of the intramedullary screw engage the screw threads in the central opening of the spiked washer, whereby to lock the intramedullary screw to the spiked washer.

In one form of the invention, there is provided a surgical implant system comprising:

a spiked washer for impacting against the proximal end of a bone so as to achieve fixed positioning relative to the proximal end of the bone, wherein the spiked washer comprises a disk-like body having a central opening, a plurality of spikes extending distally from the disk-like body, and screw threads formed in the central opening of the disk-like body; and

an intramedullary screw for advancement through the central opening of the spiked washer and down the intramedullary canal of a bone, wherein the intramedullary screw comprises a shaft and a head, distal threads formed on the shaft for engaging the wall of the intramedullary canal at the distal end of the intramedullary screw and proximal threads formed on the shaft for engaging the wall of the intramedullary canal at the proximal end of the intramedullary screw, and locking threads formed on the head for engaging the screw threads formed in the central opening of the spiked washer, whereby to lock the intramedullary screw to the spiked washer.

In another form of the invention, there is provided a method for fracture fixation, the method comprising:

providing a surgical implant system comprising:
a spiked washer for impacting against the proximal end of a bone so as to achieve fixed positioning relative to the proximal end of the bone, wherein the spiked washer comprises a disk-like body having a central opening, a plurality of spikes extending distally from the disk-like body, and screw threads formed in the central opening of the disk-like body; and
an intramedullary screw for advancement through the central opening of the spiked washer and down the intramedullary canal of a bone, wherein the intramedullary screw comprises a shaft and a head, distal threads formed on the shaft for engaging the wall of the intramedullary canal at the distal end of the intramedullary screw and proximal threads formed on the shaft for engaging the wall of the intramedullary canal at the proximal end of the intramedullary screw, and locking threads formed on the head for engaging the screw threads formed in the central opening of the spiked washer, whereby to lock the intramedullary screw to the spiked washer;

securing the spiked washer to the outer surface of a bone; and

advancing the intramedullary screw through the central opening of the spiked washer and through the bone so that the distal threads on the intramedullary screw engage a distal portion of the bone and the proximal threads on the intramedullary screw engage a proximal portion of the bone, whereby to generate a compressive force therebetween, and such that the locking threads on the head engage the screw threads formed in the central opening of the spiked washer so as to lock the intramedullary screw to the spiked washer.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing the bone structure of a human foot;

FIG. 2 is a schematic view showing the fifth metatarsal bone;

FIG. 3 is a schematic view showing an intramedullary screw being used to repair a Jones fracture in the conventional manner;

FIG. 4 is a schematic view showing the intramedullary canal of the fifth metatarsal;

FIG. 5 is a schematic view showing the novel surgical implant system of the present invention;

FIG. 6 is a schematic view showing the intramedullary screw of the novel surgical implant system shown in FIG. 5;

FIG. 7 is a schematic view showing the spiked washer of the novel surgical implant system shown in FIG. 5;

FIG. 8 is a schematic view showing the novel surgical implant system of FIG. 5 being used to repair a Jones fracture in accordance with the present invention;

FIGS. 8A, 8B and 8C are schematic views showing alternative forms of the intramedullary screw of the novel surgical implant system shown in FIG. 5; and

FIGS. 9-23 are schematic views showing other surgical procedures which may be enhanced by using the novel surgical implant system of the present invention.

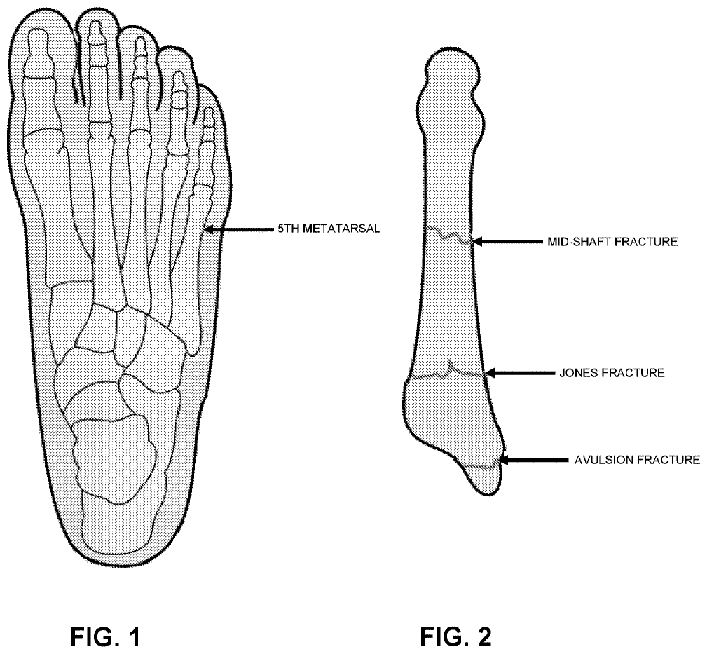

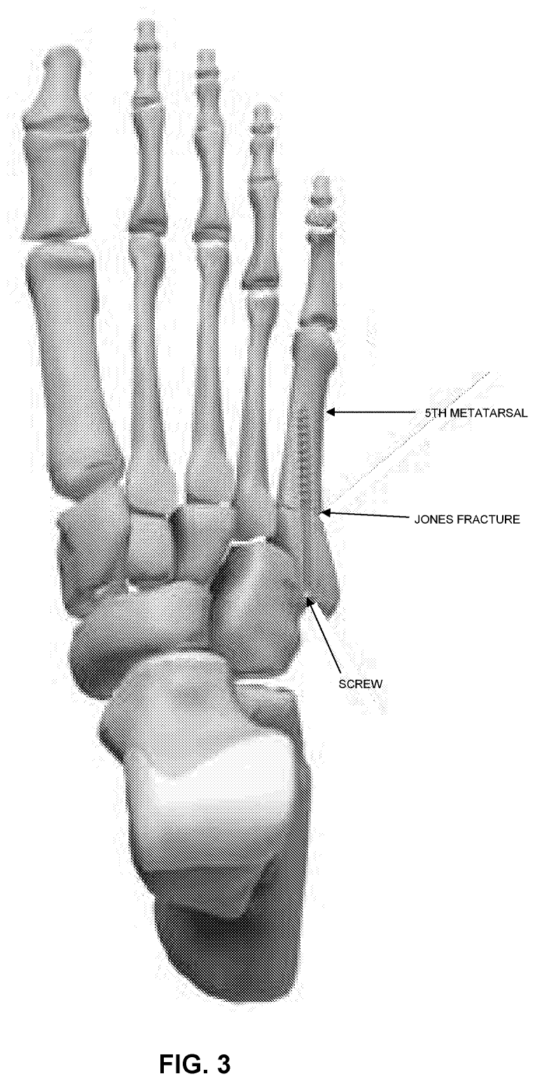

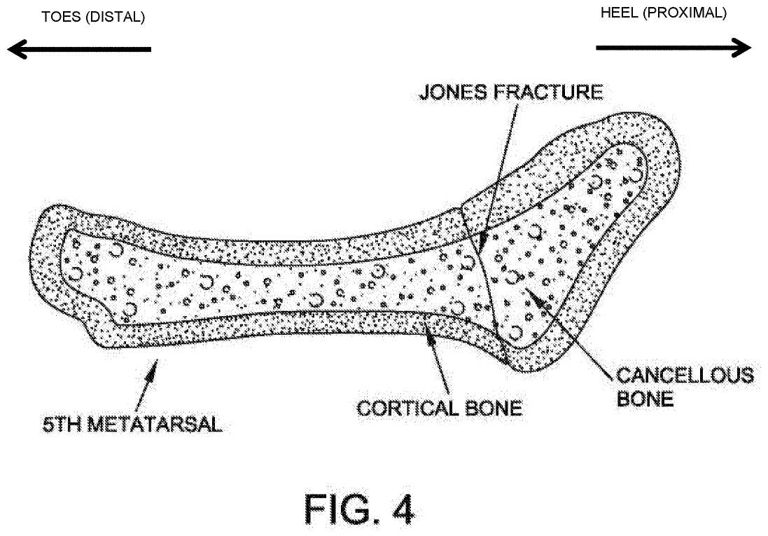

### DETAILED DESCRIPTION OF THE PREFERRED EMBODIMENTS

The present invention comprises the provision and use of a new and improved system for providing intramedullary screw fixation of a Jones fracture of the fifth metatarsal, wherein the system provides for adequate fixation of both the distal and proximal ends of the intramedullary screw.

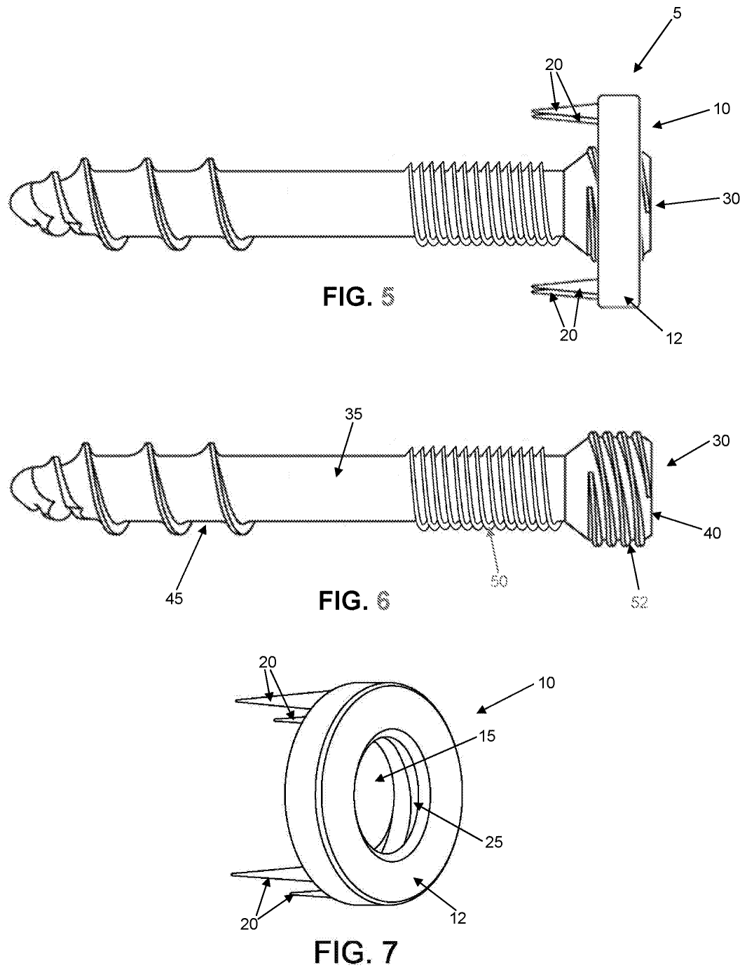

More particularly, and looking now at FIGS. 5-7, the present invention comprises a novel surgical implant system 5 comprising (i) a spiked washer 10 for impacting against the proximal end of the fifth metatarsal so as to achieve fixed positioning relative to the proximal end of the fifth metatarsal, wherein spiked washer 10 comprises a disk-like body 12 having a central opening 15, a plurality of spikes 20 extending distally from disk-like body 12, and screw threads 25 formed in central opening 15 of disk-like body 12, and (ii) an intramedullary screw 30 for advancement through central opening 15 of spiked washer 10 and down the intramedullary canal of the fifth metatarsal, wherein intramedullary screw 30 comprises a shaft 35 and a head 40, distal threads 45 formed on shaft 35 for engaging the narrowed wall of the intramedullary canal at the distal end of the intramedullary screw, and proximal threads 50 formed on shaft 35 for engaging the wall of the intramedullary canal at the proximal end of the intramedullary screw. In a preferred form of the present invention, distal threads 45 have a more aggressive pitch than proximal threads 50, whereby to generate compression (e.g., between the distal bone fragment and the proximal bone fragment) when intramedullary screw 30 is inserted into the intramedullary canal. Locking threads 52 are formed on head 40 for engaging screw threads 25 formed in central opening 15 of spiked washer 10, whereby to lock spiked washer 10 to intramedullary screw 30 when intramedullary screw 30 is fully deployed within the intramedullary canal. Intramedullary screw 30 also comprises means (not shown) in head 40 for turning intramedullary screw 30 (e.g., a hex recess, a slot recess, etc.).

As a result of this construction, distal threads 45 of intramedullary screw 30 can securely anchor the distal end of intramedullary screw 30 against the narrowed wall of the intramedullary canal, proximal threads 50 can securely anchor the proximal end of intramedullary screw 30 against the wall of the intramedullary canal (with the differential in the thread pitch between distal threads 45 and proximal threads 50 generating compressive force therebetween), and locking threads 52 of intramedullary screw 30 can engage screw threads 25 formed in central opening 15 of spiked washer 10 so as to lock intramedullary screw 30 to spiked washer 10. In essence, spiked washer 10, which is impacted into position against the proximal end of the fifth metatarsal, serves as a capture plate for holding the proximal end of intramedullary screw 30 fixed in position relative to the proximal end of the fifth metatarsal, whereby to eliminate the aforementioned “windshield wiper” effect which could otherwise occur within the wider, weaker cancellous bone of the proximal end of the fifth metatarsal. In addition, by locking intramedullary screw 30 to spiked washer 10, intramedullary screw 30 is fixed against rotation. As a result, novel surgical implant system 5 can prevent the fracture site from experiencing a substantial bending moment under stress, thereby preventing re-fracture.

In one preferred form of the invention, the distal and proximal threads 45, 50 of intramedullary screw 30 have different pitches, with distal threads 45 having a more aggressive pitch than proximal threads 50, and with an unthreaded gap extending between the distal threads and the proximal threads, whereby to create compression between the distal bone fragment and the proximal bone fragment when the intramedullary screw is inserted into the bone. Spiked washer 10 is locked to the proximal end of the fifth metatarsal and intramedullary screw 30 is deployed through central opening 15 of spiked washer 10 and down the intramedullary canal of the fifth metatarsal, with locking threads 52 of intramedullary screw 30 engaging screw threads 25 of spiked washer 10, whereby to lock intramedullary screw 30 to spiked washer 10. More particularly, in this form of the invention, the thread pitch on proximal thread 50 is finer than the thread pitch on distal thread 45 (i.e., the thread pitch on proximal thread 50 has more threads per inch than the thread pitch on distal thread 45), whereby to provide a pitch differential which aids in reducing fractures and in creating compression between bone fragments when intramedullary screw 30 is deployed through central opening 15 of spiked washer 10 and down the intramedullary canal of the fifth metatarsal.

And in one preferred form of the invention, central opening 15 of spiked washer 10 and screw threads 25 formed in central opening 15, are coordinated with locking threads 52 of intramedullary screw 30 so as to allow for variable-angle locking of intramedullary screw 30 to spiked washer 10.

And in one preferred form of the invention, spiked washer 10 can be made of stainless steel, which is more rigid, or titanium, which is more flexible, having a modulus of elasticity closer to bone. Titanium offers the additional benefit of improved bony ingrowth, which is ideal at the spike-bone interface.

And in one preferred form of the invention, intramedullary screw 30 can be made of stainless steel.

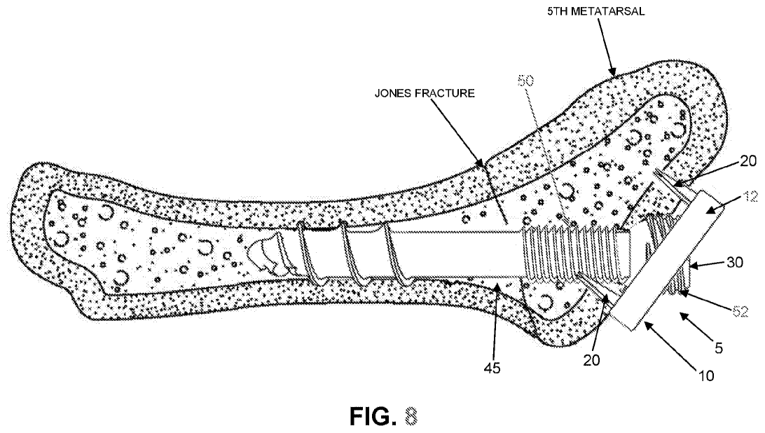

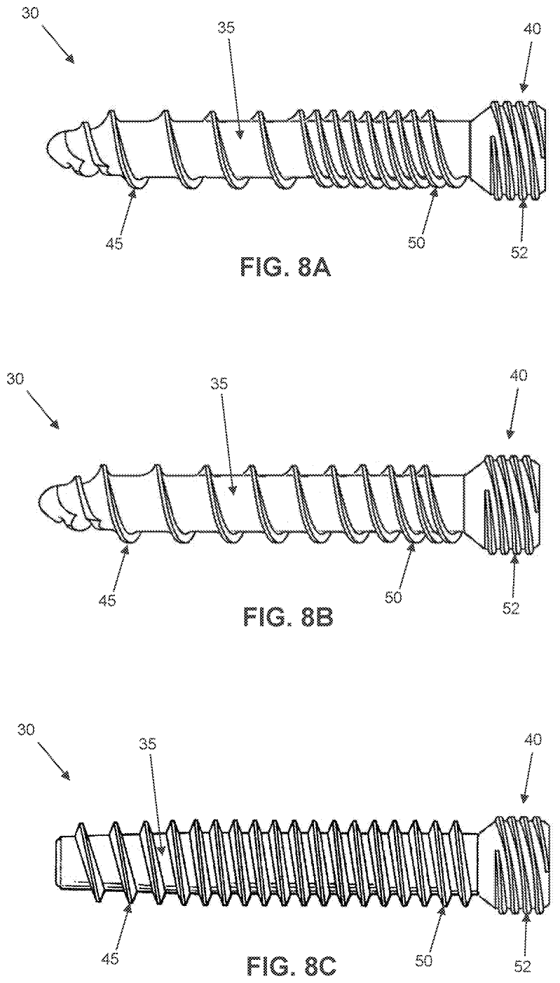

In use, and looking now at FIG. 8, spiked washer 10 is impacted into position against the proximal end of the fifth metatarsal, and then intramedullary screw 30 is advanced through central opening 15 of spiked washer 10 and down the intramedullary canal of the fifth metatarsal, such that distal threads 45 of intramedullary screw 30 and proximal threads 50 of intramedullary screws 30 engage with the wall of the intramedullary canal so as to generate a compressive force therebetween (i.e., a compressive force between the distal bone fragment and the proximal bone fragment) and locking threads 52 of intramedullary screw 30 engage with screw threads 25 in central opening 15 of spiked washer 10 so as to lock intramedullary screw 30 to spiked washer 10.

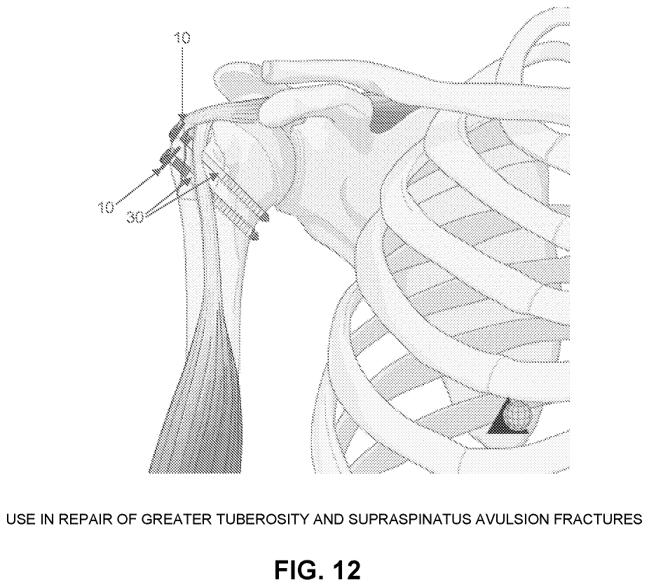

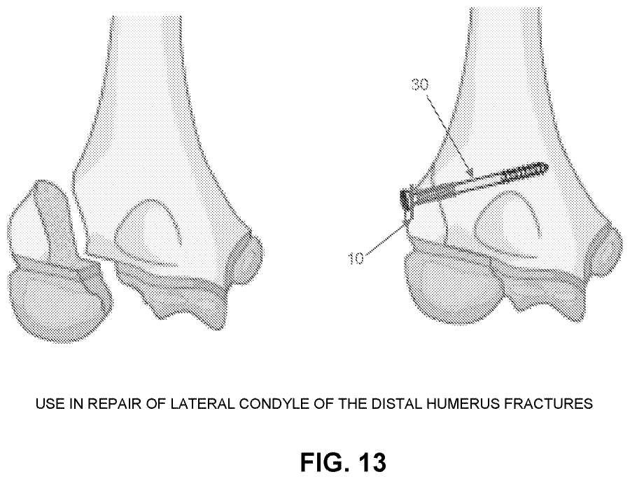

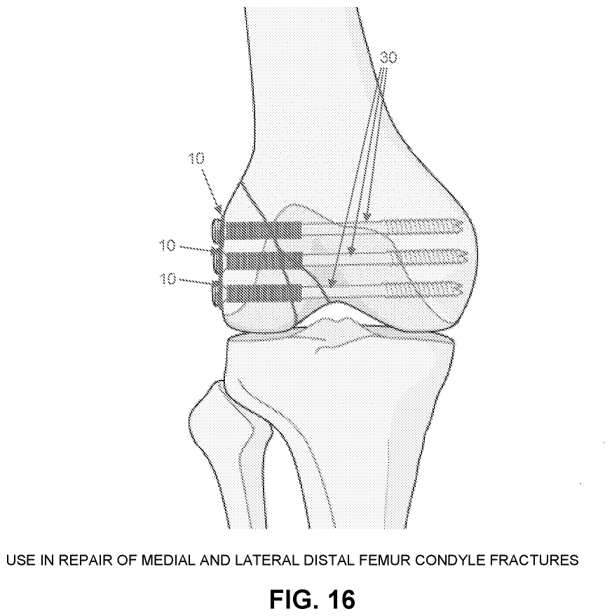

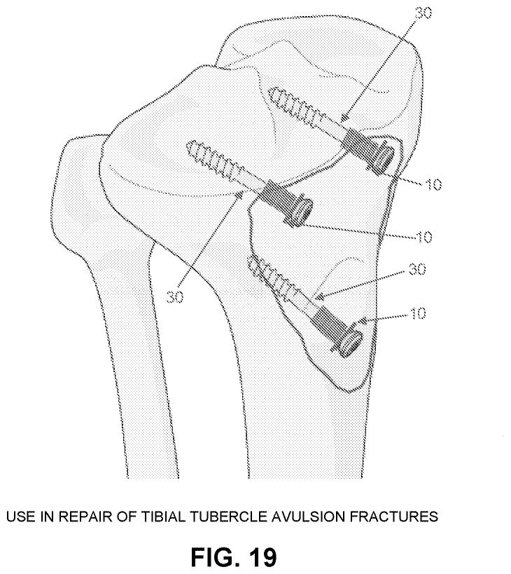

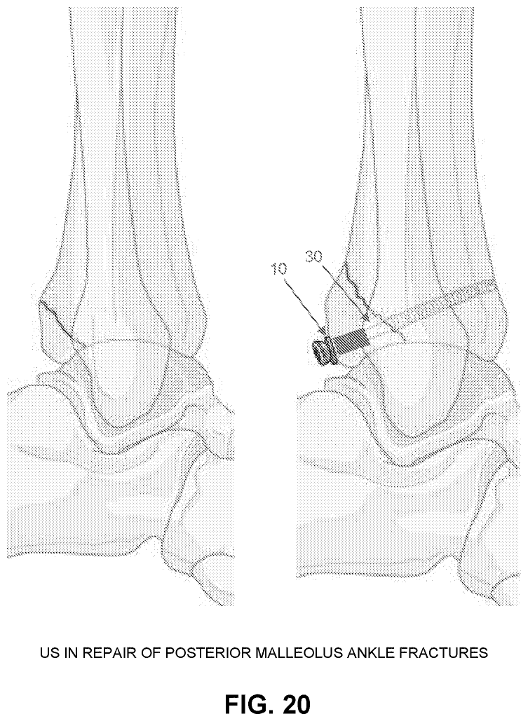

In the intramedullary screw 30 shown in FIGS. 5, 6, 8, 9, 12, 13, 15, 16 and 18-20, distal threads 45 are separated from proximal threads 50 by a gap (i.e., shaft 35 of intramedullary screw 30 is threadless along an intermediate portion of the shaft). This construction can be advantageous since it facilitates forming distal threads 45 and proximal threads 50 with different thread pitches. However, if desired, distal threads 45 and proximal threads 50 can be formed as parts of a single continuous thread, with the thread pitch varying along shaft 35 (e.g., with two discrete thread sections each having a different fixed thread pitch such as is shown in FIG. 8A, or with a thread pitch which varies at a constant rate along the length of the single continuous thread such as is shown in FIG. 8B, or with a thread pitch which varies at a variable rate along the length of the single continuous thread such as is shown in FIG. 8C, etc.).

### Additional Applications

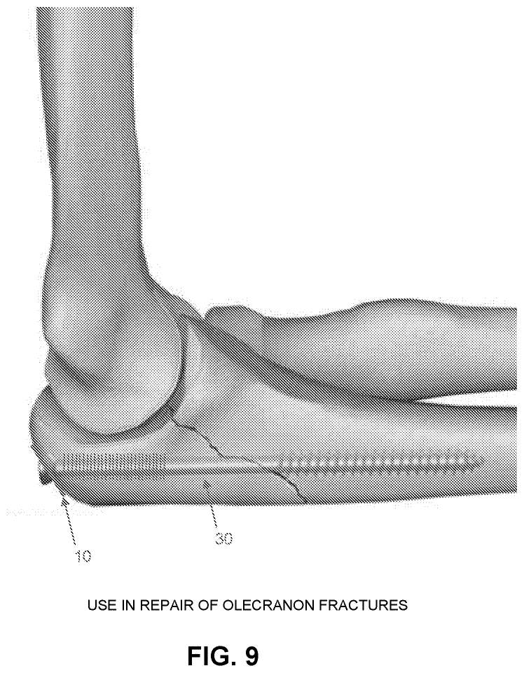

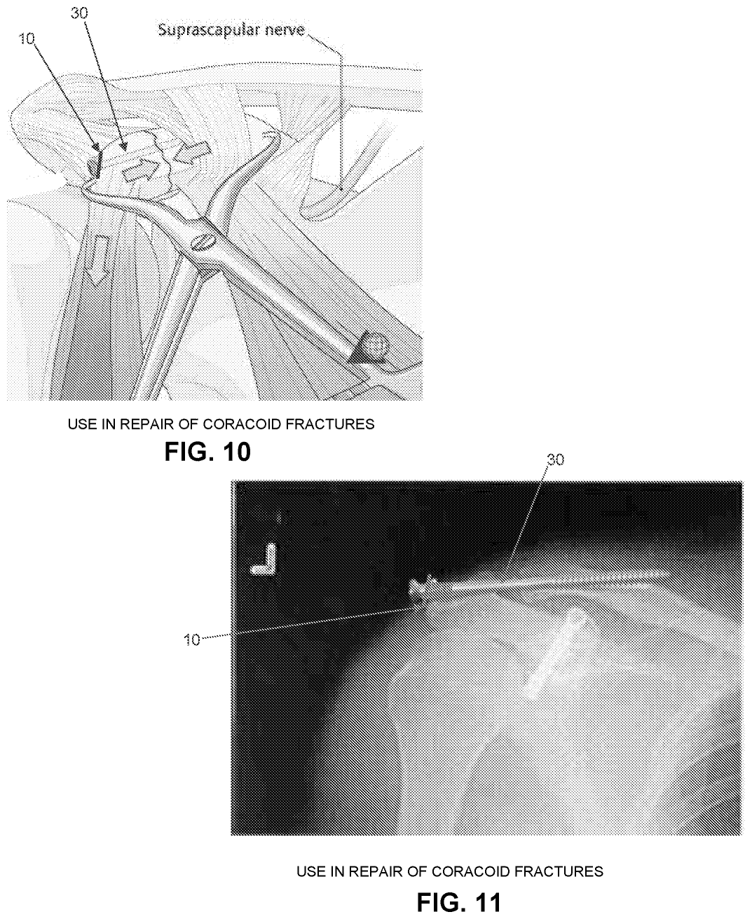

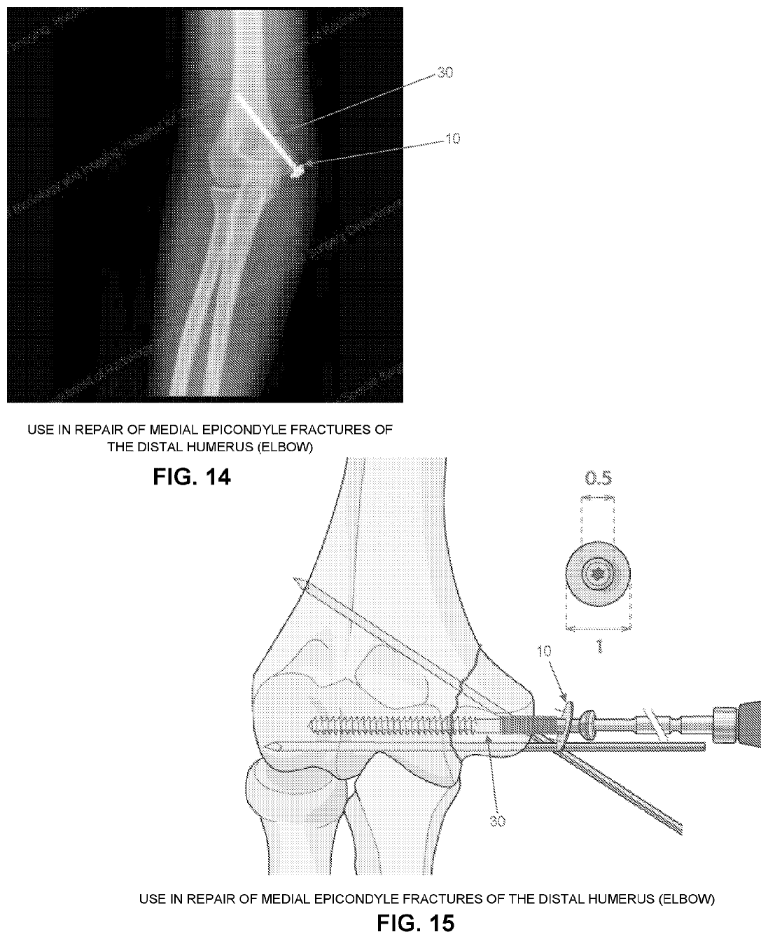

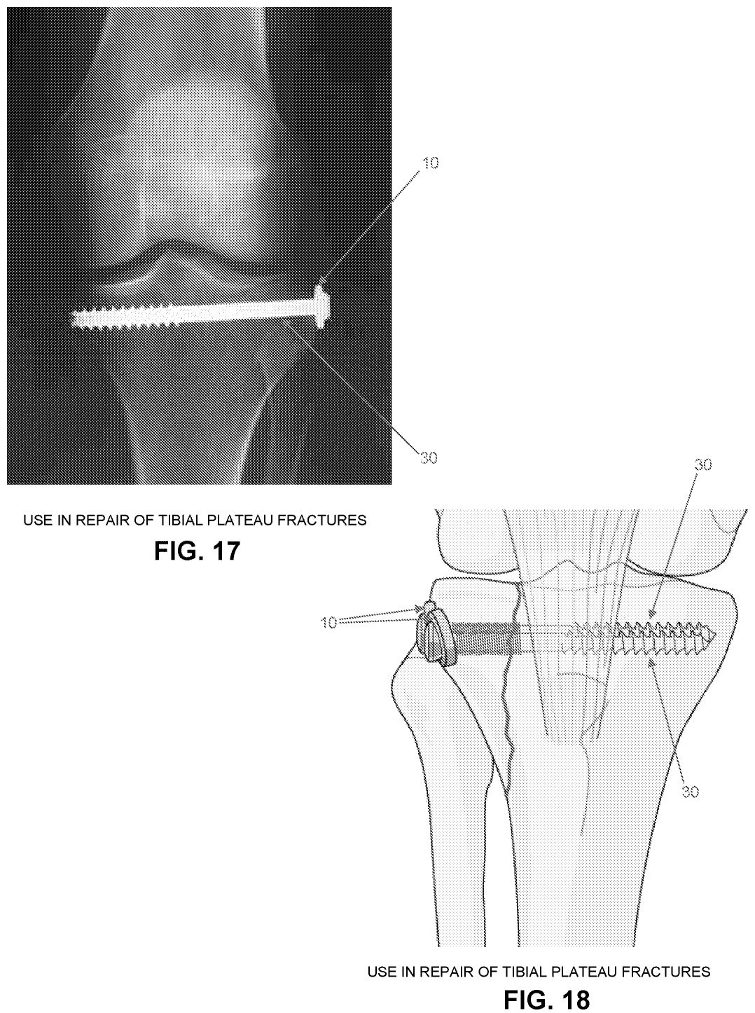

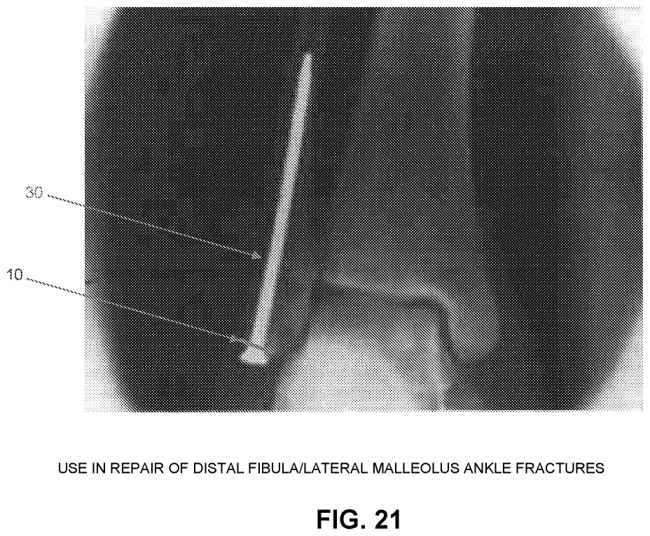

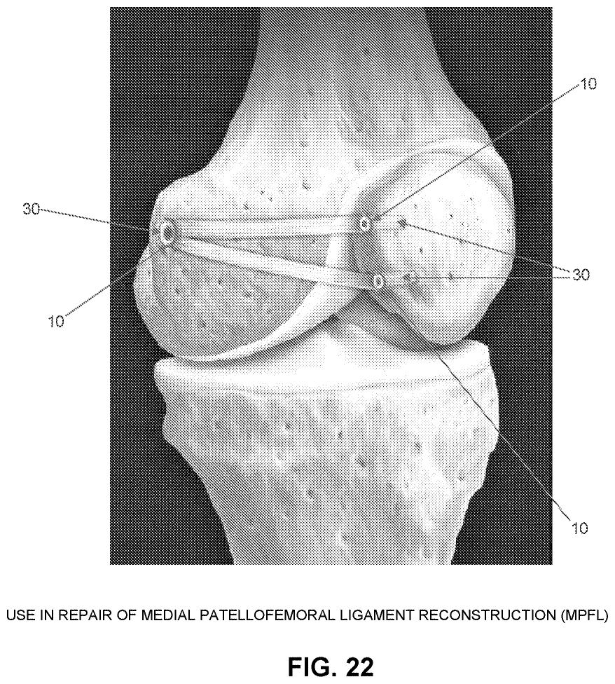

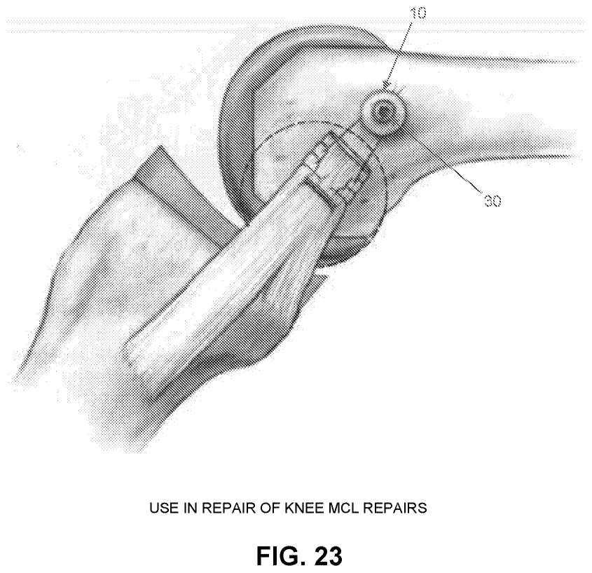

It is also possible to use the novel surgical implant system of the present invention for other applications. By way of example but not limitation, FIGS. 9-23 show a variety of surgical procedures which may be enhanced by using the novel surgical implant system of the present invention. Still other possible applications include LCL knee repairs, patellar tendon avulsions, PCL avulsion fractures, Pec tendon avulsions, etc.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A surgical implant system comprising:
a spiked washer for impacting against the proximal end of a bone so as to achieve fixed positioning relative to the proximal end of the bone, wherein the spiked washer comprises a disk-like body having a central opening, a plurality of spikes extending distally from the disk-like body, and screw threads formed in the central opening of the disk-like body; and
an intramedullary screw for advancement through the central opening of the spiked washer and down the intramedullary canal of a bone, wherein the intramedullary screw comprises a shaft and a head, distal threads formed on the shaft for engaging the wall of the intramedullary canal at the distal end of the intramedullary screw and proximal threads formed on the shaft for engaging the wall of the intramedullary canal at the proximal end of the intramedullary screw, and locking threads formed on the head for engaging the screw threads formed in the central opening of the spiked washer, whereby to lock the intramedullary screw to the spiked washer.

2. A surgical implant system according to claim 1 wherein the distal and proximal threads of the intramedullary screw have different pitches, with the distal threads having a more aggressive pitch than the proximal threads, whereby to create compression when the intramedullary screw is deployed through the central opening of the spiked washer and engages a second bone fragment.

3. A surgical implant system according to claim 2 wherein the thread pitch on the proximal thread is finer than the thread pitch on the distal thread.

4. A surgical implant system according to claim 1 wherein the central opening of the spiked washer and the screw threads formed in the central opening are coordinated with the locking threads of the intramedullary screw so as to allow for variable-angle locking of the intramedullary screw to the spiked washer.

5. A surgical implant system according to claim 1 wherein the spiked washer is made from a material selected from the group consisting of stainless steel and titanium.

6. A surgical implant system according to claim 1 wherein the intramedullary screw is made of stainless steel.

7. A surgical implant system according to claim 1 wherein the spiked washer and the intramedullary screw are sized for deployment on the fifth metatarsal bone.

8. A method for fracture fixation, the method comprising:
providing a surgical implant system comprising:
a spiked washer for impacting against the proximal end of a bone so as to achieve fixed positioning relative to the proximal end of the bone, wherein the spiked washer comprises a disk-like body having a central opening, a plurality of spikes extending distally from the disk-like body, and screw threads formed in the central opening of the disk-like body; and
an intramedullary screw for advancement through the central opening of the spiked washer and down the intramedullary canal of a bone, wherein the intramedullary screw comprises a shaft and a head, distal threads formed on the shaft for engaging the wall of the intramedullary canal at the distal end of the intramedullary screw and proximal threads formed on the shaft for engaging the wall of the intramedullary canal at the proximal end of the intramedullary screw, and locking threads formed on the head for engaging the screw threads formed in the central opening of the spiked washer, whereby to lock the intramedullary screw to the spiked washer;
securing the spiked washer to the outer surface of a bone; and
advancing the intramedullary screw through the central opening of the spiked washer and through the bone so that the distal threads on the intramedullary screw engage a distal portion of the bone and the proximal threads on the intramedullary screw engage a proximal portion of the bone, whereby to generate a compressive force therebetween, and such that the locking threads on the head engage the screw threads formed in the central opening of the spiked washer so as to lock the intramedullary screw to the spiked washer.

9. A method according to claim 8 wherein the distal and proximal threads of the intramedullary screw have different pitches, with the distal threads having a more aggressive pitch than the proximal threads, whereby to create compression when the intramedullary screw is deployed through the central opening of the spiked washer and engages a second bone fragment.

10. A method according to claim 9 wherein the thread pitch on the proximal thread is finer than the thread pitch on the distal thread.

11. A method according to claim 8 wherein the central opening of the spiked washer and the screw threads formed in the central opening are coordinated with the locking threads of the intramedullary screw so as to allow for variable-angle locking of the intramedullary screw to the spiked washer.

12. A method according to claim 8 wherein the spiked washer is made from a material selected from the group consisting of stainless steel and titanium.

13. A method according to claim 8 wherein the intramedullary screw is made of stainless steel.

14. A method according to claim 8 wherein the spiked washer and the intramedullary screw are deployed on the fifth metatarsal bone.
